(19) United States
(12) Patent Application Publication
(10) Pub. No.: US 2025/0382571 A1
(43) Pub. Date: Dec. 18, 2025

(54) PRODUCING METHOD OF ASSEMBLOIDS AND COMPUTING DEVICES COMPRISING THE ASSEMBLOIDS

(71) Applicants: SAMSUNG ELECTRONICS CO., LTD., Suwon-si (KR); Korea University Research and Business Foundation, Seoul (KR)

(72) Inventors: Anes Ju, Suwon-si (KR); Woong Sun, Seoul (KR); Seo-Kyung Kong, Seoul (KR); Takhyung Lee, Suwon-si (KR)

(73) Assignees: SAMSUNG ELECTRONICS CO., LTD., Suwon-si (KR); Korea University Research and Business Foundation, Seoul (KR)

(21) Appl. No.: 19/007,243

(22) Filed: Dec. 31, 2024

(30) Foreign Application Priority Data
Jun. 14, 2024 (KR) 10-2024-0077722

(51) Int. Cl. C12N 5/0793; C12N 5/00; G01N 27/327

(52) U.S. Cl. CPC C12N 5/0619; C12N 5/0068; G01N 27/327; C12N 2506/03; C12N 2513/00; C12N 2527/00; C12N 2533/90

(57) ABSTRACT

A method of producing an assembloid, including: producing a plurality of necrotic core-free organoids; producing a primary assembloid having at least one of a sheet shape and a line shape by connecting the plurality of necrotic core-free organoids; and producing a secondary assembloid by laterally or vertically connecting the primary assembloid with at least one additional primary assembloid.

### CROSS-REFERENCE TO RELATED APPLICATION

This application is based on and claims priority under 35 U.S.C. § 119 to Korean Patent Application No. 10-2024-0077722 filed on Jun. 14, 2024, in the Korean Intellectual Property Office, the disclosure of which is incorporated by reference herein in its entirety.

### BACKGROUND

### 1. Field

The disclosure relates to a method of producing assembloids, and a computing device including the assembloids.

### 2. Description of the Related Art

An organoid may refer to a small culture that reproduces both the form and function of a tissue or an organ by coagulating and recombining cells which are isolated from stem cells or organ origin cells through three-dimensional culture. These organoids may include several specific cell populations that may construct organs or tissues. Organoids may have a similar form to actual tissues or organs and may be structurally organized. Organoids may reproduce a special function of each organ. Organoids may be formed through a series of common processes. Cells with the same function may be grouped together and put in an appropriate position. After the cells are separated into compartments, a more detailed differentiation may occur.

A neural organoid (NO) may be a three-dimensional cell aggregate produced through the self-organization ability of pluripotent stem cells. The NO may mimic the structure and function of a nervous system through a process similar to the development of the nervous system during an embryonic development process. These characteristics may allow NOs to be used as a model system for human disease research and biomedical applications. In addition, because it may be difficult to obtain living human brain tissue, NOs may be used for various engineering attempts.

Accordingly, organoids may be used for computing by attaching NOs that implement neural circuits similar to a brain to a multi-electrode array (MEA). As a result, there is need for new organoid production technology to implement highly integrated neural circuits has been underlined.

### SUMMARY

In accordance with an aspect of the disclosure, a method of producing an assembloid includes: producing a plurality of necrotic core-free organoids; producing a primary assembloid having at least one of a sheet shape and a line shape by connecting the plurality of necrotic core-free organoids; and producing a secondary assembloid by laterally or vertically connecting the primary assembloid with at least one additional primary assembloid.

The producing the plurality of necrotic core-free organoids may include: culturing a stem cell in a cell culture substrate; and separating, from the cell culture substrate, an organoid that is differentiated from the stem cell.

The cell culture substrate may include Matrigel.

A diameter of each of the plurality of necrotic core-free organoids may be less than or equal to 1 millimeter.

The primary assembloid may be formed in a single layer in which the plurality of necrotic core-free organoids are combined.

The primary assembloid may be produced using an orbital shaker after the plurality of necrotic core-free organoids are positioned in a well.

The primary assembloid may be produced by adjusting at least one of a size of each of the plurality of necrotic core-free organoids, a size of the well, and a rotation speed of the orbital shaker.

A diameter of the well may be in a range from 35 mm corresponding to a 6-well plate, to 15.5 mm, corresponding to a 24-well plate, and a rotation speed of the orbital shaker may be in a range from 30 rotations per minute (RPM) to 300 RPM.

The primary assembloid may have the sheet shape, and the secondary assembloid may be produced by stacking a plurality of layers including a plurality of sheet-shaped primary assembloids.

The plurality of sheet-shaped primary assembloids may be fused using Matrigel embedding.

The primary assembloid may have the line shape, and the secondary assembloid may be produced by arranging a plurality of line-shaped assembloids in parallel.

The secondary assembloid may be formed by combining a plurality of primary assembloids having different shapes.

In accordance with an aspect of the disclosure, a computing device includes: an assembloid, wherein the assembloid includes a secondary assembloid including a plurality of stacked layers, wherein each layer of the plurality of stacked layers includes a plurality of sheet-shaped primary assembloids to which a plurality of organoids is connected.

Each organoid of the plurality of organoids may be a necrotic core-free organoid having a diameter that is less than or equal to 1 millimeter (mm).

The secondary assembloid may be on a multi-electrode array (MEA).

The computing device may include at least one from among a biocomputer, a neuromorphic semiconductor, a biosensor, a biomimetic organ chip, and a quantum computer.

In accordance with an aspect of the disclosure, a computing device includes: an assembloid, wherein the assembloid includes a secondary assembloid, wherein the secondary assembloid includes a plurality of line-shaped assembloids arranged in parallel, and wherein a plurality of organoids is connected to the plurality of line-shaped assembloids.

The secondary assembloid may be a single layer assembloid.

Each organoid of the plurality of organoids may include a necrotic core-free organoid having a diameter that is less than or equal to 1 millimeter (mm), and the secondary assembloid may be on a multi-electrode array (MEA).

The computing device may include at least one from among a biocomputer, a neuromorphic semiconductor, a biosensor, a biomimetic organ chip, and a quantum computer.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a flowchart illustrating a method of producing an assembloid, according to one or more embodiments;

FIG. 2 is a diagram illustrating the concepts of primary assembly (top) and secondary assembly (bottom) of an assembloid in a method of producing an assembloid, according to one or more embodiments;

FIG. 3 is a diagram illustrating a secondary assembly of an assembloid using Matrigel embedding in a method of producing an assembloid, according to one or more embodiments;

FIG. 4A is a diagram illustrating a two-dimensional assembloid stacked in a sheet shape on a two-dimensional multi-electrode array (MEA), according to one or more embodiments;

FIG. 4B is a diagram illustrating a two-dimensional assembloid stacked in a line shape on a two-dimensional MEA, according to one or more embodiments;

FIG. 5 is a diagram illustrating a method of controlling the shape of sheet-shaped and line-shaped assembloids by using the size of a well and an orbital shaker, according to one or more embodiments;

FIG. 6 is a diagram illustrating processes of primary assembly and secondary assembly in a method of producing an assembloid, according to one or more embodiments.

FIG. 7 is a diagram illustrating each zone according to a distance from the center of a well.

FIG. 8A is a graph illustrating the results of controlling a movement direction of an organoid with a diameter of 500 micrometers (μm) by adjusting the size of a well and a rotation speed of an orbital shaker, according to one or more embodiments.

FIG. 8B is a graph illustrating the results of controlling a movement direction of an organoid with a diameter of 1000 μm by adjusting the size of a well and a rotation speed of an orbital shaker, according to one or more embodiments.

FIG. 9A is a picture illustrating a position of an organoid when a 500 μm-sized organoid is rotated at a speed of 80 rotations per minute (RPM) in a 24-well plate (e.g., in a well having a diameter of 15.50 millimeters (mm)), according to one or more embodiments.

FIG. 9B is a picture illustrating a position of an organoid when a 500 μm-sized organoid is rotated at a speed of 80 RPM in a 12-well plate (e.g., in a well having a diameter of 21.90 mm), according to one or more embodiments.

FIG. 9C is a picture illustrating a position of an organoid when a 1000 μm-sized organoid is rotated at a speed of 100 RPM in a 24-well plate, according to one or more embodiments.

FIG. 9D is a picture illustrating a position of an organoid when a 1000 μm-sized organoid is rotated at a speed of 100 RPM in a 12-well plate, according to one or more embodiments.

FIG. 10 is a diagram illustrating a bundle of exons extending to a secondarily assembled assembloid, according to one or more embodiments.

FIG. 11A is a sample picture to confirm the stability of attachment of a single organoid to an MEA.

FIG. 11B is a sample picture to confirm the stability of attachment of an assembloid to an MEA.

FIG. 12 is a graph illustrating a ratio of a pixel area to a sample area by comparing the single organoid of FIG. 11A and the assembloid of FIG. 11B.

FIG. 13A is a diagram illustrating a general spherical organoid disposed on an MEA.

FIG. 13B is a diagram illustrating a sheet-shaped assembloid according to one or more embodiments disposed on an MEA.

### DETAILED DESCRIPTION

Hereinafter, embodiments are described in detail with reference to the accompanying drawings. When describing the embodiments with reference to the accompanying drawings, like reference numerals refer to like elements and a repeated description related thereto will be omitted.

The term “organoid” as used herein may refer to a three-dimensional structure that has a structure, cellular components, and/or functions similar to those of a living organ (e.g., an organ in a human body).

The term “computing device” as used herein may encompass electronic devices that may perform functions such as inputting, calculating, learning, processing, storing, and outputting data. For example, a computing device may include at least one from among neuromorphic computing devices, including a neuron, a synapse, or a spiking neural network (SNN), and von Neumann structured computing devices, including a central processing unit (CPU), a memory, a storage, or an input/output device, capable of data processing and operations. The neuromorphic computing devices may mimic the structure and function of a human brain and may process and learn data in a similar manner to that of the human nervous system. In addition, for example, the computing device may include at least one from among a deoxyribonucleic acid (DNA)-based or cell-based bio-computing device, a biosensor using the characteristics of responding to an external stimulus, a biomimetic organ-on-a-chip, which mimics the functions of a human organ, and a quantum computing device.

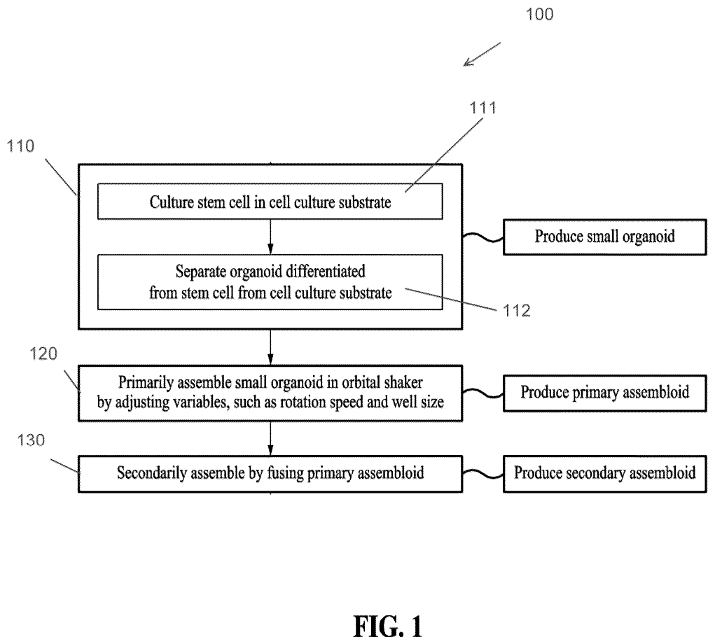

FIG. 1 is a flowchart illustrating a method of producing an assembloid, according to one or more embodiments. Referring to FIG. 1, the assembloid, according to an embodiment, may be produced by sequentially producing a small organoid at operation 110, producing a primary assembloid at operation 120, and producing a secondary assembloid at operation 130.

Specifically, at operation 110, the producing of the small organoid may include culturing a stem cell in a cell culture substrate at operation 111, and separating an organoid differentiated from the stem cell from the cell culture substrate at operation 112. According to one or more embodiments, a stem cell that may be used to produce the small organoid may refer to a cell having pluripotency, which may refer to the potential to differentiate into cells derived from the endoderm, mesoderm, and ectoderm of animals, or multipotency, which may refer to the potential to differentiate into cells closely related to a tissue or a function.

The culturing of the stem cell in the cell culture substrate at operation 110 may induce a phenotype of a specific cell or the differentiation from the stem cell to the specific cell.

The cell culture substrate used in the culturing of the stem cell may include at least one from among a biologically derived matrix, such as fibrin, collagen, and Matrigel, and a synthetic hydrogel, such as polyacrylamide (PAA) and polyethylene glycol (PEG). According to one or more embodiments, Matrigel may provide a cell culture environment similar to living tissue (which may provide biocompatibility), may be formed in a gel state at 37° Celsius (C) (which may provide temperature sensitivity), and may include growth factors to be described below.

The Matrigel may include, as a protein complex extracted from sarcoma cells of an Engelbreth-Holm-Swarm (EHS) mouse, at least one from among an extracellular matrix (ECM), such as laminin, collagen, or heparan sulfate proteoglycan, and growth factors, such as a fibroblast growth factor (FGF), an epiderma growth factor (EFG), an insulin-like growth factor (IGF), a transforming growth factor-beta (TGF-β), and a platelet-derived growth factor (PDGF).

In such a cell culture substrate as described above, the stem cell may be cultured to form a cell colony, and then, the cell colony may be differentiated to produce an organoid. By separating it from the cell culture substrate, a small organoid may be finally obtained.

According to one or more embodiments, the produced small organoid may be a necrotic core-free organoid. A necrotic core-free organoid may refer to an organoid having no cell necrosis at a three-dimensional center of the organoid. As the size of an organoid increases, it may become difficult to supply oxygen and nutrients to the center, which may result in the formation of a necrotic core. Thus, a size (e.g., a diameter) of the organoid may be less than or equal to 1000 micrometers (μm. For example, the diameter may be less than or equal to 500 μm or 250 μm, and the lower limit of the diameter may be any size as desired. However, when comprehensively considering a contact area with an MEA to be described below, the flatness of the MEA, and the formation of a necrotic core, the diameter may preferably be less than or equal to 500 μm.

In addition, the small organoid may be derived from organoids from different brain regions, such as at least one of the cerebrum, midbrain, and cerebellum. By connecting the organoids from different brain regions and producing primary and secondary assembloids, a complex having a complex nervous system, similar to a human brain, may be formed.

According to one or more embodiments, a method of producing an assembloid may include connecting a plurality of organoids and producing a primary assembloid. For example, a sheet-shaped or line-shaped primary assembloid may be produced by rotating the plurality of organoids in a device, such as an orbital shaker, which may refer to a device for rotating a sample in a circular orbit by adjusting variables, like rotation speed and well size.

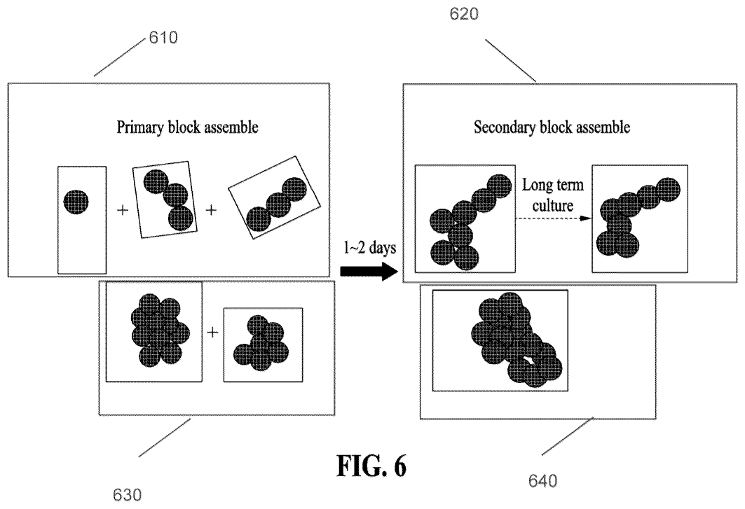

Here, the sheet-shaped primary assembloid may refer to an assembloid having a surface in a specific shape with all the plurality of organoids being connected to one another, as illustrated portion 630 illustrated in FIG. 6, without the plurality of organoids being connected in series.

In addition, the line-shaped assembloid may refer to an assembloid having a shape in which the plurality of organoids may be connected in series as illustrated in portion 610 illustrated in of FIG. 6.

Accordingly, the primary assembloid may be formed in a single layer into which the plurality of organoids may be combined.

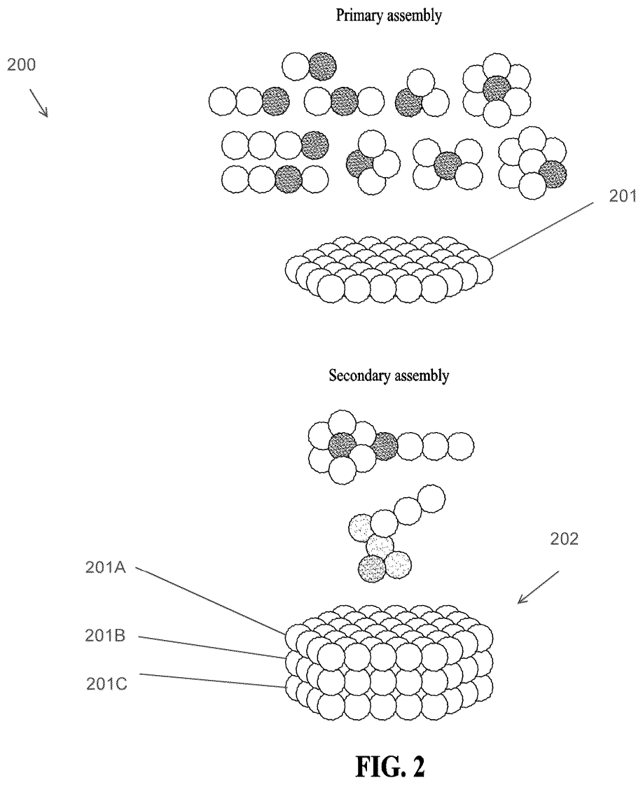

FIG. 2 conceptually illustrates an example of an assembly process of a sheet-shaped primary assembloid 201 and a secondary assembloid 202 in which sheet-shaped primary assembloids 201 (e.g., a first primary assembloid 201A, a second primary assembloid 201B, and a third primary assembloid 201C) are stacked in three layers, according to one or more embodiments. As shown in an experimental example described in more detail below, FIG. 6 illustrates an assembly process of a primary assembloid and an unstandardized secondary assembloid, according to one or more embodiments.

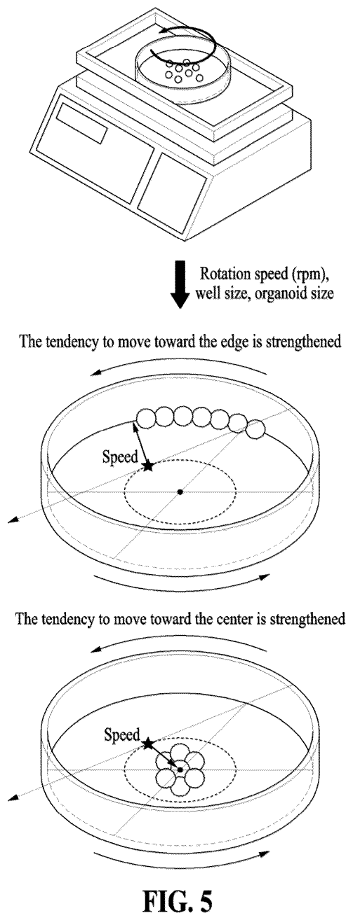

According to one or more embodiments, to produce the primary assembloid having a specific shape, such as a sheet shape or a line shape, the size of an organoid, the size of a well in which the organoid is disposed, and the rotation speed of the orbital shaker may be adjusted. As illustrated in FIG. 5, as the rotation speed (e.g., the rotations per minute (RPM)) decreases, the size of the well may decrease (e.g., may decrease from a 6-well plate to a 24-well plate), and the size of the organoid gets larger, the tendency to move to the edge is intensified due to a fluid flow by rotation. However, as the rotation speed increases, the size of the well may increase (e.g., may increase from a 24-well plate to a 6-well plate), and the size of the organoid may decrease, the tendency to move to the center may be intensified. Therefore, when the tendency to move to the edge is intensified, the plurality of organoids may be connected in series along the edge of the well and the line-shaped primary assembloid may be produced. Conversely, when the tendency to move to the center is intensified, the plurality of organoids may be disposed in the center of the well and the sheet-shaped primary assembloid may be produced.

According to one or more embodiments, when producing a primary assembloid, as described above, when a small organoid less than or equal to 1 mm is used, the size of the well may correspond to a 6-well plate or a 24-well plate, and the rotation speed of the orbital shaker may be greater than or equal to 30 RPM and less than or equal to 300 RPM, but embodiments are not limited thereto.

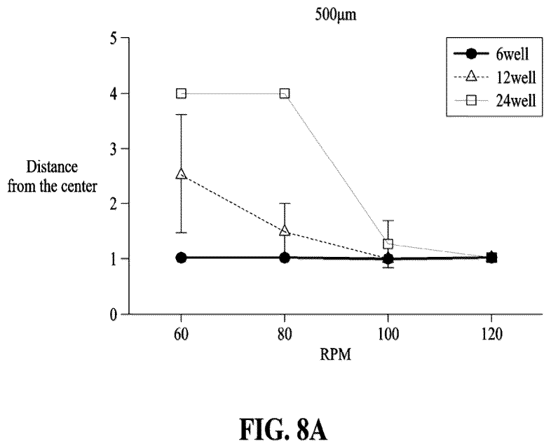

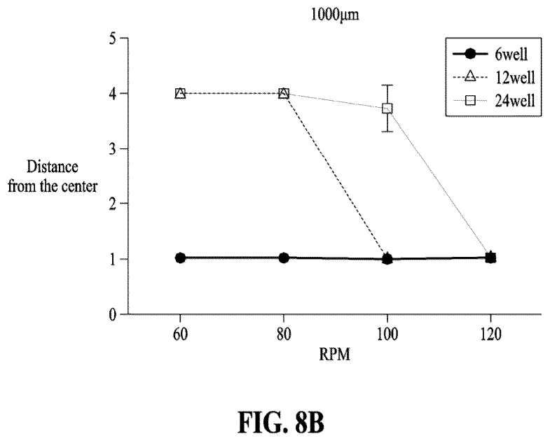

For example, as illustrated in FIG. 8A, when using an organoid having a size (e.g., a diameter) of 500 μm, by using a 6-well plate or a rotation speed exceeding 80 RPM in 12-well plate or a 24-well plate and intensifying the tendency to move to the center, the sheet-shaped primary assembloid may be produced. As another example, to produce a line-shaped primary assembloid, a rotation speed less than or equal to 80 RPM in a 24-well plate or a rotation speed less than 80 RPM in a 12-well plate may be used.

In addition, when using a rotation speed that is less than or equal to about 80 RPM, primary assembloids in different shapes may be distinctively produced for the same organoid only by varying the well size. For example, when using the rotation speed that is less than or equal to approximately 80 RPM, primary assembloids in a line shape may be produced by using a 24-well plate, and primary assembloids in a sheet shape may be produced by using a 6-well plate or a 12-well plate, but embodiments are not limited thereto.

As illustrated in FIG. 8B, when using an organoid having a size (diameter) of 1000 μm, by using a 6-well plate, a rotation speed exceeding 80 RPM in a 12-well plate, or a rotation speed exceeding 100 RPM in a 24-well plate, and intensifying the tendency to move to the center of the organoid, the sheet-shaped primary assembloid may be produced. Conversely, to provide the line-shaped primary assembloid, a rotation speed less than or equal to 80 RPM in a 12-well plate and less than or equal to 100 RPM in a 24-well plate may be used.

In addition, when using a rotation speed that is in a range of about 90 to 110 RPM, primary assembloids in different shapes may be distinctively produced for the same organoid only by varying the well size. For example, when using the rotation speed that is in the range of about 90 to 110 RPM, primary assembloids in a line shape may be produced by using a 24-well plate, and primary assembloids in a sheet shape may be produced by using a 6-well plate or a 12-well plate, but embodiments are not limited thereto.

According to at least one embodiment, a method of producing an assembloid may include connecting the primary assembloids and producing a secondary assembloid. The secondary assembloid may be assembled by laterally or vertically fusing the produced primary assembloids.

When the primary assembloids are sheet-shaped, the secondary assembloid may be produced by stacking two or more layers of the sheet-shaped primary assembloids.

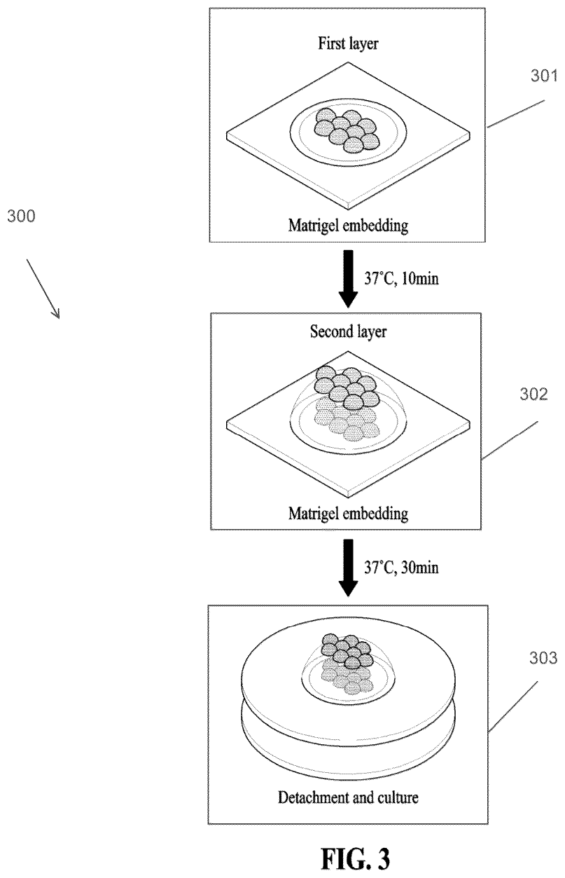

FIG. 3 is a diagram illustrating a secondary assembly of an assembloid using Matrigel embedding in a method of producing an assembloid, according to one or more embodiments. According to one or more embodiments, as illustrated in FIG. 3, a process 300 may include culturing a sheet-shaped primary assembloid as a first layer at operation 301 using Matrigel embedding. Then, at operation 302, using Matrigel embedding, a second-layer sheet-shaped assembloid on the first-layer sheet-shaped assembloid and interlayer fusion may be performed. Then, the secondary assembloid, which may be vertically stacked in multiple layers may be produced at operation 303. The interlayer fusion of the secondary assembloid may be readily performed. Unless a problem occurs in an access rate of a neural network, the number of stacks is not particularly limited, and, for example, may be less than or equal to 100 layers, 50 layers, or 10 layers, according to one or more embodiments. By producing an assembloid in such a shape, an access rate of a three-dimensional neural network having a greater size than that of a typical neural network may increase.

According to one or more embodiments, when the primary assembloids are line-shaped, the line-shaped assembloids may be arranged in parallel such that the primary assembloids may be laterally fused. By producing an assembloid in such a shape, the assembloid may simulate the connectivity in a neuro-module unit in a brain.

Although examples are described above in which the primary assembloids have a sheet shape or line shape, embodiments are not limited thereto. For example, in some embodiments, primary assembloids each having different shapes may be combined to implement the shape of a secondary assembloid having a complex structure that is more complex than the structure of the above-described shapes. The complex structure may refer to a structure in which two or more sheets may be stacked, a structure in which two or more lines may be arranged in parallel, and other unstandardized three-dimensional structures.

An assembloid produced according to one or more embodiments may be used as a computing device.

When using the assembloid as a computing device, the assembloid produced according to one or more embodiments may be disposed on a two-dimensional MEA.

As described above in detail, the assembloid may include a secondary assembloid in which there is stacked two or more layers of sheet-shaped primary assembloids to which a plurality of organoids is connected. In addition, in production method of an assembloid according to one or more embodiments, by adjusting the size of an organoid, the size of a well, and the rotation speed of an orbital shaker, an assembloid including a secondary assembloid in which line-shaped primary assembloids to which a plurality of organoids is connected may be arranged in parallel.

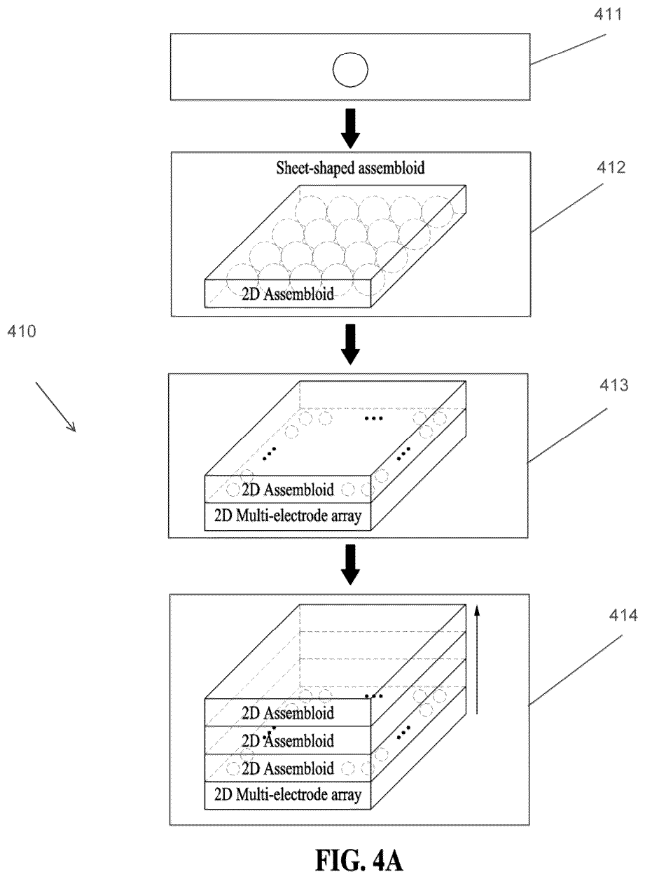

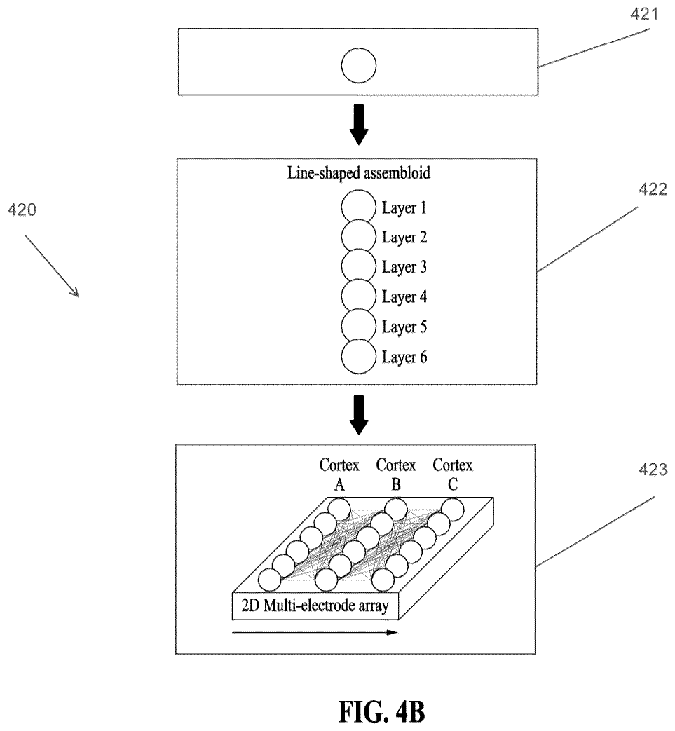

FIG. 4A and FIG. 4B are diagrams each illustrating a process for creating an assembloid one or more embodiments disposed on a two-dimensional MEA, according to one or more embodiments.

According to a process 410 illustrated in FIG. 4A, Matrigel, which may be a material for attaching a cell, may be coated on the two-dimensional MEA, and a necrotic core-free organoid may be provided at operation 411. Then, at operation 412, a sheet-shaped primary assembloid may be attached for a relatively long time such that the first-layer assembloid may be disposed on the MEA. Then, sheet-shaped second-layer assembloid, which may be the same as or similar to the first-layer assembloid, may be disposed on the first-layer assembloid in the same manner at operation 413. By repeating operation 413, the process 410 may provide an MEA on which may be disposed three-dimensional assembloids having the desired number of stacks or layers. The assembloid, according to one or more embodiments, may have a higher attachment stability and connection rate for the two-dimensional MEA compared to a single spherical organoid.

In addition, as an example, FIG. 4B illustrates a process 420 in which a secondary assembloid is produced using line-shaped primary assembloid. At operation 421, a necrotic core-free organoid may be provided, and then at operation 422 line-shaped primary assembloids may be produced. Then, at operation 423, a plurality of the line-shaped primary assembloids may be arranged in parallel and the primary assembloids may be laterally fused, being disposed on the two-dimensional MEA. According to one or more embodiments, the secondary assembloid may be formed as a single layer. Through this secondary assembloid, the connectivity of a neuro-module unit in a brain may be simulated.

According to one or more embodiments, the computing device may include a biocomputer, a neuromorphic semiconductor, a biosensor, a biomimetic organ chip, or a quantum computer.

According to one or more embodiments, the biocomputer may be a computer which may be capable of using the unique characteristics of a biological system beyond a typical silicon-based computer. For example, the biocomputer may include at least one of a cell-based computing device for manipulating a genetic circuit and biochemical reaction in a cell using a living cell and performing a logical operation, a protein-based computing device for using a reaction of an enzyme or a specific binding of proteins and performing a logical operation, and a DNA computing device for using a base pair of DNAs to store and process information. When using the assembloid, according to one or more embodiments, as a biocomputer, DNA molecules in cells of the assembloid may be manipulated or the cells of the assembloid may be designed to have a specific reaction under a specific condition such that complex operations may be performed by using the assembloid.

The neuromorphic semiconductor, according to one or more embodiments, may be a semiconductor which may mimic the structure and function of a human brain, beyond a computing device having a general-purpose computer structure of a von Neumann structure capable of processing and operating data, including a CPU, a memory, a storage, or an input/output device. The neuromorphic semiconductor may include a chip or an artificial synapse, such as a human neural network, which may simulate neurons or synapses. Because the neurons may not consume power in an inactive state, the neuromorphic semiconductor may have low heat dissipation and significantly low power consumption.

The neuromorphic semiconductor may include a structure similar to a three-dimensional semiconductor integrated circuit. For example, a three-dimensional assembloid may be produced by stacking sheet-shaped assembloids to increase an access rate of a neural network according to one or more embodiments, an assembloid suitable for the neuromorphic semiconductor may be a secondary assembloid in which two or more layers of sheet-shaped primary assembloids are stacked.

The biosensor, according to one or more embodiments, may implement functions as a sensor by using the characteristics of specific cells responding to external stimuli. Because cells included in an organoid may generate or react to electrical signals, the assembloid, according to one or more embodiments, may also be used as a component of the biosensor.

The biomimetic organ chip, according to one or more embodiments, may be a chip which may simulate the functions of human organs. The biomimetic organ chip may be used to test (toxicity evaluation, etc.) how organs of a human body respond to various diseases or drugs. The biomimetic organ chip may require high biocompatibility and precision. An assembloid fused by arranging, in parallel, line-shaped assembloids that simulate the connectivity in a neuro-module unit in a brain, or an assembloid in a structure in which multiple layers of sheet-shaped assembloids are stacked to implement a three-dimensional neural network where an access rate between organoids is increased, may all be used as components suitable for the biomimetic organ chip.

The quantum computer, according to one or more embodiments, is a complex biological system implemented as a quantum computing device, and the assembloids, according to one or more embodiments, may be combined to be used as the quantum computing device in that the quantum computer may solve complex operations with low power, like the computing device described above.

Examples of assembloids according to one or more embodiments are discussed in greater detail below.

FIG. 6 is a diagram illustrating primary assembly and secondary assembly in a method of producing an assembloid, according to one or more embodiments. The example shown in FIG. 6 may be referred to as Experimental Example 1, which may relate to secondary fusion of assembloids.

As shown in portion 610 of FIG. 6, neural organoids having a diameter of approximately 500 μm cultured for 30 days were primarily assembled in a 24-well plate to produce a line-shaped primary assembloid including three organoids. Two primary assembloids and a single organoid were secondarily assembled in a 12-well plate, and a secondary assembloid of a new pattern was formed, as shown in portion 610 of FIG. 6 . . . . In response to continuous culturing under the same rotation condition, the established pattern was maintained, and it was confirmed that stronger fusion was formed.

As shown in portion 630 of FIG. 6, ten neural organoids having a diameter of approximately 400 μm cultured for 25 days were primarily assembled in a 12-well plate, and six neural organoids of the same size were primarily assembled in a 24-well plate to produce two types of sheet-shaped primary assembloids. As shown in portion 640 of FIG. 6, these were reassembled again in a 12-well plate, and a secondary assembloid of the same pattern in which two primary assembloids are combined was formed.

Through Experimental Example 1, it may be confirmed that various shapes of primary assembloids may be produced only by differentiating the well size and the number of organoids under the same condition. As a result, it was confirmed that a three-dimensional assembloid of an unstandardized complex structure in a desired shape was produced, besides primary assembloids of the same sheet shape and line shape, by combining primary assembloids of different shapes.

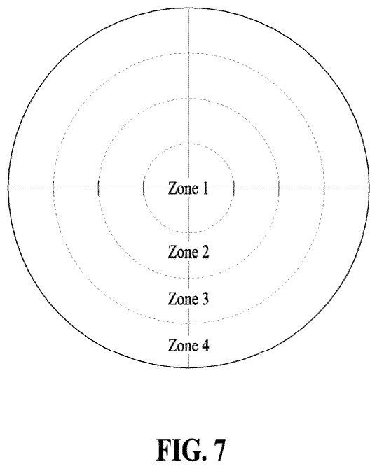

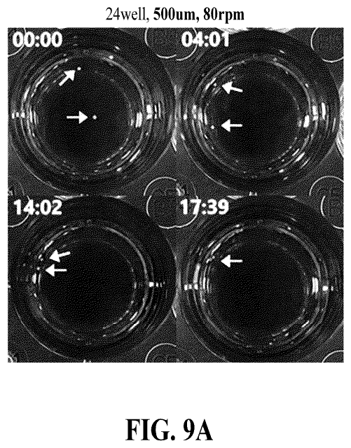

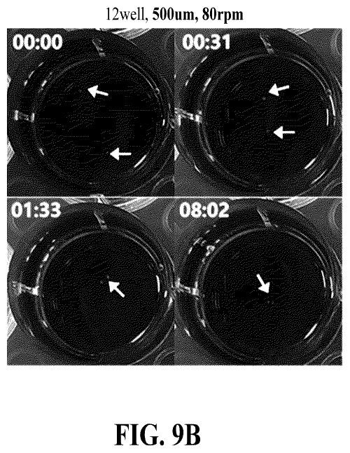

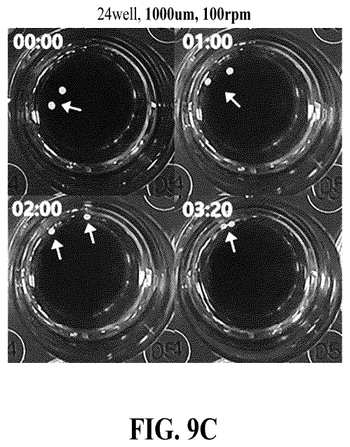

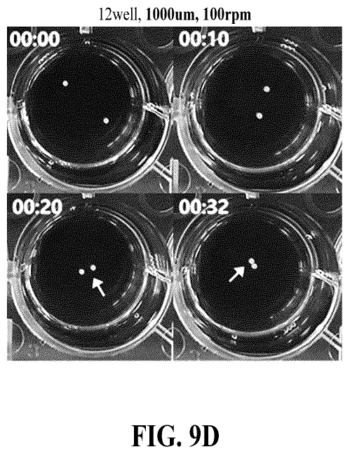

FIG. 7 is a diagram illustrating a plurality of zones according to a distance from the center of a well. FIGS. 8A-8B are graphs illustrating the results of controlling a movement direction of an organoid by adjusting the size of a well and a rotation speed of an orbital shaker, according to one or more embodiments. FIGS. 9A-9D are pictures illustrating positions of organoids under different conditions, according to one or more embodiments. The example shown in FIGS. 7, 8A-8B, and 9A-9D may be referred to as Experimental Example 2, which may relate to shape control of sheet-shaped and line-shaped assembloids by fluid flow.

According to Experimental Example 2, the movement patterns of individual organoids under four different conditions were tracked by adjusting the rotation speed of an orbital shaker, a well size, and the size of organoids as below by using the orbital shaker (N-biotek, NB101SRC; Orbit diameter: 22 mm), as specified below:
Orbital shaker rotation speed: 60 RPM, 80 RPM, 100 RPM, or 120 RPM;
Well size: 6-well plate (e.g., 35 mm in diameter), 12-well plate (e.g., 21.90 mm in diameter), or 24-well plate (e.g., 15.50 mm in diameter); and
Organoid size: 500 μm in diameter or 1000 μm in diameter.

After one to two hours of observation, a specific region that finally stagnated was confirmed. A well was divided into four regions from the center of the well to the edge of the well, and the regions were designated as Zone 1, Zone 2, Zone 3, and Zone 4, according to their distances from the center, as illustrated in FIG. 7.

The results are shown in the graphs of FIGS. 8A and 8B, and the positions of organoids in the well over time were confirmed as illustrated in FIGS. 9A to 9D. In particular, FIG. 8A illustrates the results of controlling a movement direction of an organoid with a diameter of 500 μm, and FIG. 8B is a graph illustrating the results of controlling a movement direction of an organoid with a diameter of 1000 μm, according to one or more embodiments. FIG. 9A illustrates a position of an organoid when a 500 μm-sized organoid is rotated at a speed of 80 RPM in a 24-well plate (e.g., in a well having a diameter of 15.50 mm), according to one or more embodiments. FIG. 9B is a picture illustrating a position of an organoid when a 500 μm-sized organoid is rotated at a speed of 80 RPM in a 12-well plate (e.g., in a well having a diameter of 21.90 mm), according to one or more embodiments. FIG. 9C is a picture illustrating a position of an organoid when a 1000 μm-sized organoid is rotated at a speed of 100 RPM in a 24-well plate, according to one or more embodiments. FIG. 9D is a picture illustrating a position of an organoid when a 1000 μm-sized organoid is rotated at a speed of 100 RPM in a 12-well plate, according to one or more embodiments.

Through Experimental Example 2, as the well size increased (increased from 24-well plate to 6-well plate), the rotation speed increased, or the organoid size decreased, the tendency to move to the center was intensified. Organoids with a diameter of about 500 μm at 80 RPM had a stronger tendency to move toward the edge in 24 wells and move toward the center in 1a 12-well plate. Organoids with a diameter of about 1000 μm at 100 RPM had a stronger tendency to move toward the edge in a 24-well plate and to move toward the center in a 12-well plate. It was confirmed that the position of an individual organoid was controlled by adjusting the well size when a rotation speed value was fixed.

Accordingly, when multiple organoids were put in a well, it was predicted that the organoids would be combined in a line shape along the edge, or the organoids were combined in a sheet shape when gathering at the center. Actually, when putting 30-day-old organoids with a diameter of about 500 μm at 80 RPM in different wells and culturing the organoids for 24 to 30 hours in an orbital shaker, a sheet-shaped assembloid was produced in 6-well plates and 12-well plates, and a line-shaped assembloid was produced in a 24-well plates.

For example, it was confirmed that the shape and size of a primary assembloid to which organoids had been fused may be homogeneously produced by adjusting the size of organoids, the size of wells, and the rotation speed of orbital shakers under a certain condition, or a primary assembloid to which organoids had been fused were controlled by differently adjusting the size of organoids, the size of wells, and the rotation speed of orbital shakers.

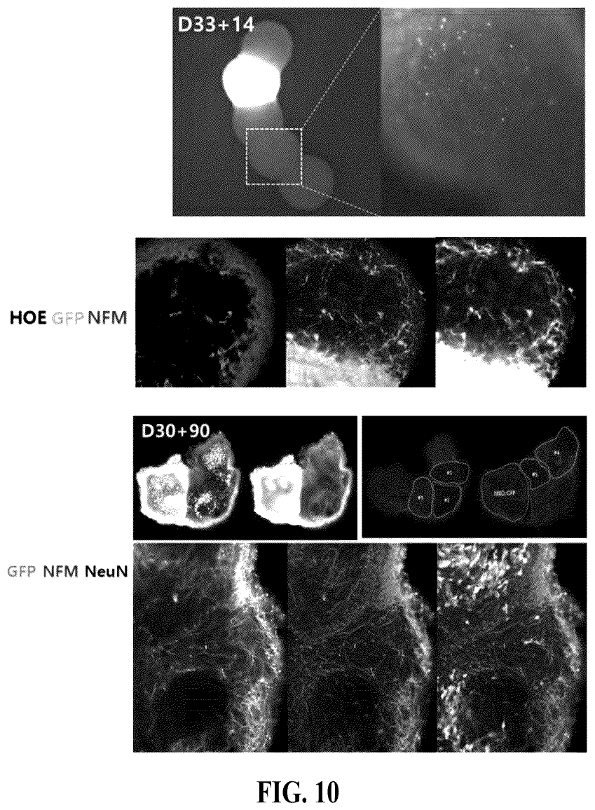

FIG. 10 is a diagram illustrating a bundle of exons extending to a secondarily assembled assembloid, according to one or more embodiments. The example shown in FIG. 10 may be referred to as Experimental Example 3, which may relate to formation of neural circuit in an assembloid.

In fused organoids produced through a green fluorescent protein (GFP) cell line, by imaging a GFP fluorescent expression through an optical microscope (EVOS), a growth path of organoid-derived exons was examined. As a result, in sequentially connected assembloids, it was confirmed that GFP organoid-derived exons extended to the inside of an organoid after the next, besides a directly neighboring organoid.

As a result of imaging a GFP fluorescent marker and a mature neuron marker NFM through immunofluorescent staining by using a confocal microscope (Leica TCS SP8), it was confirmed that GFP-derived exons were extended at a significant level in a sample cultured long term for three months after fusion. In other words, the identity of a GFP organoid section was maintained and a connection to another neural organoid through exon bundles was confirmed.

Along a previously established neural circuit path of a neural organoid, it was shown that exons derived from the outside grew while interacting with an internal histological structure.

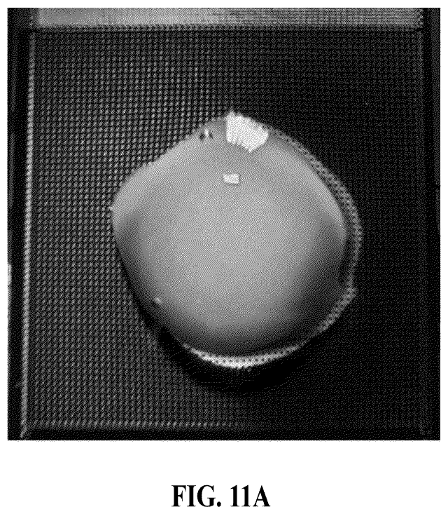

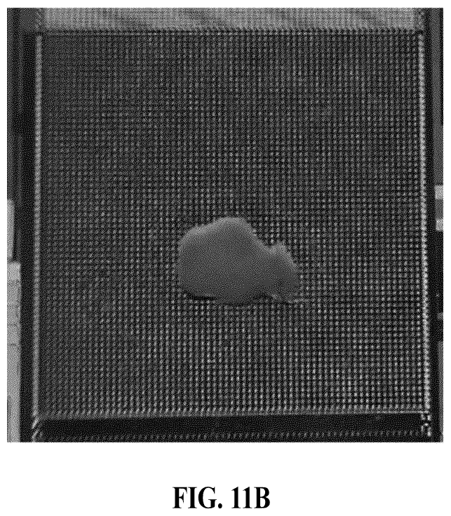

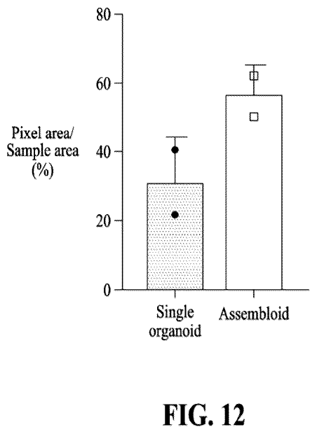

FIG. 11A is a sample picture to confirm the stability of attachment of a single organoid to an MEA. FIG. 11B is a sample picture to confirm the stability of attachment of an assembloid to an MEA. FIG. 12 is a graph illustrating a ratio of a pixel area to a sample area by comparing the single organoid of FIG. 11A and the assembloid of FIG. 11B. The examples shown in FIGS. 11A-11B and 12 may be referred to as Experimental Example 4, which may relate to improvement of attachment stability of mea of sheet-shaped assembloid.

The attachment stability of a wide and flat sheet-shaped assembloid with a height less than or equal to 0.3 mm and a width less than or equal to 2 mm, which may be produced by using an assembloid formation principle through the control of a fluid flow and the attachment stability of a general spherical single organoid with a diameter less than or equal to 2 mm, were tested under the same condition while being attached to a planar MEA. The spherical organoid was detached when replacing a culture medium on the next day, but the sheet-shaped assembloid was stably attached for the subsequent days of culture and even during the replacement of a culture medium.

In addition, as shown in FIG. 12, a pixel area of the MEA contacting a sample compared to a sample area was measured, and it was confirmed that the assembloid according to one or more embodiments shows an area ratio higher than that of the single organoid by 50% or more.

These results showed that the problem of organoids not being stably attached may be improved when measuring the organoids through a planar MEA. In addition, the results also showed that the complexity of an organoid sample measurable in a limited area (e.g., 3.8 mm×3.8 mm) of an MEA increased, and neural signal generation in an organoid unit may be measured.

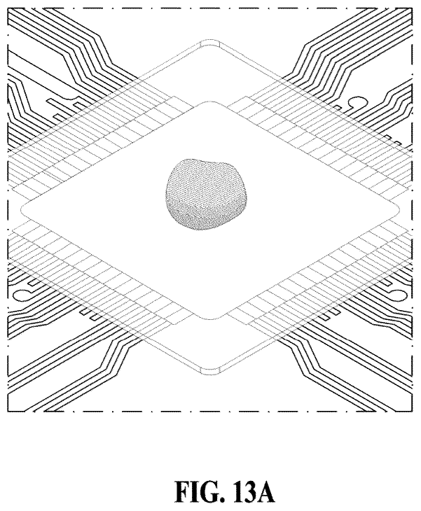

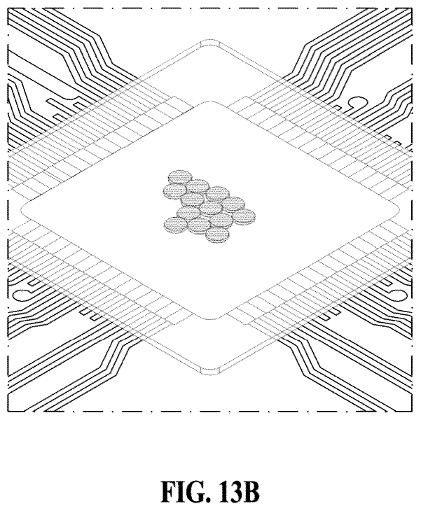

FIG. 13A is a diagram illustrating a general spherical organoid disposed on an MEA. FIG. 13B is a diagram illustrating a sheet-shaped assembloid according to one or more embodiments disposed on an MEA. According to one or more embodiments, a wide and flat surface area may be used to efficiently connect neural organoids to a two-dimensional MEA. Experimental Examples 1 to 4 discussed above may show that the producing method of an assembloid according to one or more embodiments may be used to fuse multiple neural organoids in a small size, oxygen and a medium may be supplied to the inside of the assembloid, and a desired shape may be implemented such that a wide and flat surface area may be easily formed. Accordingly, a connection rate to the two-dimensional MEA may be improved. Furthermore, by connecting various types of neural organoids, a connection to an adjacent brain region beyond a local brain region may be implemented in vitro such that a complex neural network may be reproduced.

According to the method of producing an assembloid according to one or more embodiments, the size of organoids, a well size, and the rotation speed of an orbital shaker may be adjusted under certain conditions to homogeneously control the shape and size of a primary assembloid to which organoids have been fused, and primary assembloids in the same shape may be stacked or arranged in parallel to produce a secondary assembloid in a desired, standardized shape.

According to one or more embodiments, the size of organoids, the well size, and the rotation speed of an orbital shaker may be differently adjusted to produce various shapes of primary assembloids to which organoids have been fused, and a secondary assembloid in a complex structure having the variety of shapes may be formed by pertinently fusing the primary assembloids.

Through the step-by-step assembly as described above, assembloids may be variously produced in desired patterns and arrangements, a complex neural network may be consistently implemented by using such assembloids, and a neural circuit in an organoid unit may be readily designed.

It should be understood that various embodiments of the present disclosure and the terms used therein are not intended to limit the technological features set forth herein to particular embodiments and include various changes, equivalents, or replacements for a corresponding embodiment. In connection with the description of the drawings, like reference numerals may be used for similar or related components. It should be understood that a singular form of a noun corresponding to an item may include one or more of the things, unless the relevant context clearly indicates otherwise. As used herein, “A or B”, “at least one of A and B”, “at least one of A or B”, “A, B or C”, “at least one of A, B and C”, and “A, B, or C,” each of which may include any one of the items listed together in the corresponding one of the phrases, or all possible combinations thereof. Terms such as “first”, “second” or “first” or “second” may simply be used to distinguish the component from other components in question, and do not limit the components in other aspects (e.g., importance or order).

Unless otherwise defined, all terms including technical and scientific terms used herein may have the same meaning as commonly understood by one of ordinary skill in the art to which the embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

When describing the embodiments with reference to the accompanying drawings, like reference numerals refer to like components and a repeated description related thereto will be omitted. In the description of embodiments, detailed description of well-known related structures or functions will be omitted when it is deemed that such description will cause ambiguous interpretation of the present disclosure.

In addition, terms such as first, second, A, B, (a), (b), and the like may be used to describe components of the embodiments. These terms are used only for the purpose of discriminating one component from another component, and the nature, the sequences, or the orders of the components are not limited by the terms. When one component is described as being “connected,” “coupled,” or “attached” to another component, it should be understood that one component may be connected or attached directly to another component, and an intervening component may also be “connected,” “coupled,” or “attached” to the components.

A component, which has the same common function as a component included in any one embodiment, will be described by using the same name in other embodiments. Unless explicitly described to the contrary, the configuration disclosed in any one embodiment may be applied to other embodiments, and the specific description of the repeated configuration will be omitted.

The effects to be achieved are not limited to those described above, and other effects not mentioned above will be clearly understood by one of ordinary skill in the art from this document.

What is claimed is:

1. A method of producing an assembloid, the method comprising:
producing a plurality of necrotic core-free organoids;
producing a primary assembloid by connecting the plurality of necrotic core-free organoids, the primary assembloid having at least one of a sheet shape and a line shape; and
producing a secondary assembloid by laterally or vertically connecting the primary assembloid with at least one additional primary assembloid.

2. The method of claim 1, wherein the producing the plurality of necrotic core-free organoids comprises:
culturing a stem cell in a cell culture substrate; and
separating, from the cell culture substrate, an organoid that is differentiated from the stem cell.

3. The method of claim 2, wherein the cell culture substrate comprises Matrigel.

4. The method of claim 1, wherein a diameter of each of the plurality of necrotic core-free organoids is less than or equal to 1 millimeter.

5. The method of claim 1, wherein the primary assembloid is formed in a single layer in which the plurality of necrotic core-free organoids are combined.

6. The method of claim 1, wherein the producing the primary assembloid comprises:
producing the primary assembloid using an orbital shaker after the plurality of necrotic core-free organoids are positioned in a well.

7. The method of claim 6, wherein the producing the primary assembloid comprises:
producing the primary assembloid by adjusting at least one of a size of each of the plurality of necrotic core-free organoids, a size of the well, and a rotation speed of the orbital shaker.

8. The method of claim 6, wherein a diameter of the well is in a range from 35 mm, corresponding to a 6-well plate, to 15.5 mm, corresponding to a 24-well plate, and
wherein a rotation speed of the orbital shaker is in a range from 30 rotations per minute (RPM) to 300 RPM.

9. The method of claim 1, wherein the primary assembloid has the sheet shape, and
wherein the secondary assembloid is produced by stacking a plurality of layers comprising a plurality of sheet-shaped primary assembloids.

10. The method of claim 9, wherein the plurality of sheet-shaped primary assembloids are fused using Matrigel embedding.

11. The method of claim 1, wherein the primary assembloid has the line shape, and
wherein the secondary assembloid is produced by arranging a plurality of line-shaped assembloids in parallel.

12. The method of claim 1, wherein the secondary assembloid is produced by combining a plurality of primary assembloids having different shapes.

13. A computing device comprising:
an assembloid,
wherein the assembloid comprises a secondary assembloid comprising a plurality of stacked layers,
wherein each layer of the plurality of stacked layers comprises a plurality of sheet-shaped primary assembloids, each of which comprises a plurality of connected organoids.

14. The computing device of claim 13, wherein each organoid of the plurality of organoids is a necrotic core-free organoid having a diameter that is less than or equal to 1 millimeter.

15. The computing device of claim 13, wherein the secondary assembloid is disposed on a multi-electrode array.

16. The computing device of claim 13, wherein the computing device comprises at least one from among a biocomputer, a neuromorphic semiconductor, a biosensor, a biomimetic organ chip, and a quantum computer.

17. A computing device comprising:
an assembloid,
wherein the assembloid comprises a secondary assembloid,
wherein the secondary assembloid comprises a plurality of line-shaped assembloids arranged in parallel, each of which comprises a plurality of connected organoids.

18. The computing device of claim 17, wherein the secondary assembloid is a single layer assembloid.

19. The computing device of claim 17, wherein each organoid of the plurality of organoids comprises a necrotic core-free organoid having a diameter that is less than or equal to 1 millimeter (mm), and
wherein the secondary assembloid is on a multi-electrode array (MEA).

20. The computing device of claim 17, wherein the computing device comprises at least one from among a biocomputer, a neuromorphic semiconductor, a biosensor, a biomimetic organ chip, and a quantum computer.
